(19) United States
(12) Patent Application Publication
(10) Pub. No.: US 2023/0272325 A1
(43) Pub. Date: Aug. 31, 2023

(54) MULTI-POSITION SUPPORTS FOR CELL CULTURE APPARATUSES

(71) Applicant: CORNING INCORPORATED, CORNING, NY (US)

(72) Inventors: Thomas Albert Cloutier, Arundel, ME (US); Gregory Roger Martin, Acton, ME (US)

(21) Appl. No.: 18/018,202

(22) PCT Filed: Jul. 14, 2021
(86) PCT No.: PCT/US2021/041539

§ 371 Date: Jan. 26, 2023

Related U.S. Application Data
(60) Provisional application No. 63/056,913, filed on Jul. 27, 2020.

(51) Int. Cl. C12M 3/00; C12M 1/00; C12M 1/12; C12M 3/06

(52) U.S. Cl. CPC C12M 23/48; C12M 23/50; C12M 25/06; C12M 27/14

(57) ABSTRACT

A multi-position support for a multi-layer cell culture apparatus includes a primary base that rests against a support member in an upright configuration. A support surface is offset vertically from the primary base in the upright configuration. The support surface supports the multi-layer cell culture apparatus with the multi-layer cell culture apparatus located thereon. An intermediate surface extends between the primary base and the support surface. The intermediate surface meets the primary base at an interface that extends at an oblique angle to sides of the primary base. The multi-position support has a tilted configuration where the multi-position support is rotated about the interface such that the support surface is closer to the support member than in the upright configuration with the support surface supporting the multi-layer cell culture apparatus thereon.

### CROSS-REFERENCE TO RELATED APPLICATIONS

This application claims the benefit of priority under 35 U.S.C. § 119 of U.S. Provisional Application Ser. No. 63/056,913 filed on Jul. 27, 2020, the content of which is relied upon and incorporated herein by reference in its entirety.

### FIELD

The present disclosure relates to multi-position supports for cell culture apparatuses and, in particular, multi-position supports having both an upright configuration and a tilted configuration.

### BACKGROUND

Many types of cell culture articles are constructed to provide stacked or stackable units for culturing cells. For example, T-flasks are typically made to have flat top and bottom surfaces that allow T-flasks to be stacked, providing space savings. Some modified T-flasks have multiple parallel culture surfaces within the flask to reduce time and effort associated with filling and emptying. Other culture apparatuses are multi-component assemblies having a plurality of parallel or stacked culture surfaces. With most of such stacked culture assemblies, each culture layer is isolated to reduce hydrostatic pressure on the lower culture layers. As the number of stacked layers increases, the potential effect of hydrostatic pressure increases.

One exemplary cell culture article is Corning's HYPERStack™ system. The HYPERStack™ system includes multiple modules formed of individual stackette layers that can be interconnected by flexible tubes that connect to tube connectors. The modules are interconnected for filling and emptying the HYPERStack™ system. Valves and other devices may be used to control fluid flow into and out of the HYPERStack™ system. The use of these valves and other devices can be cumbersome and provide potential leak locations.

Current processes for filling and emptying the HYPERStack™ system are inconsistent because the filling and emptying protocols involve tilting the HYPERStack™ system at various stages in order to yield better results. Without an accessory to drive this protocol, users have resorted to using whatever is on hand in the lab, such as tubing clamps, tube racks, doorstops, etc. What is needed is a multi-position support that can be used to handle cell culture apparatuses and reliably place them at multiple tiled angles during fill and empty procedures.

### BRIEF SUMMARY

In a first aspect, a multi-position support for a multi-layer cell culture apparatus comprises a primary base that rests against a support member in an upright configuration; a support surface that is offset vertically from the primary base in the upright configuration, the support surface supporting the multi-layer cell culture apparatus with the multi-layer cell culture apparatus located thereon; and an intermediate surface that extends between the primary base and the support surface, wherein the intermediate surface meets the primary base at an interface that extends at an oblique angle to sides of the primary base; wherein the multi-position support has a tilted configuration where the multi-position support is rotated about the interface such that the support surface is closer to the support member than in the upright configuration with the support surface supporting the multi-layer cell culture apparatus thereon.

According to a second aspect, there is provided the multi-position support of aspect 1, further comprising a secondary base that rests against the support member in the upright configuration.

According to a third aspect, there is provided the multi-position support of aspect 2, wherein the support surface is a first support surface, the multi-position support further comprising a second support surface located between the primary base and the secondary base, the second support surface supporting the multi-layer cell culture apparatus with the multi-layer cell culture apparatus located thereon.

According to a fourth aspect, there is provided the multi-position support of aspect 3, wherein the first support surface and the second support surface lie in substantially a same plane that is oblique to the primary base.

According to a fifth aspect, there is provided the multi-position support of aspect 4 further comprising a handle that extends outward from the secondary base and comprising a support flange that engages a fill side of the multi-layer cell culture apparatus to constrain the multi-layer cell culture apparatus on the first and second support surfaces.

According to a sixth aspect, there is provided the multi-position support of aspect 5 further comprising another support flange that extends outward from the first support surface that engages a rear side of the multi-layer cell culture apparatus that is opposite the fill side.

According to a seventh aspect, there is provided the multi-position support of any of aspects 4-6, wherein the second support surface is spaced from the support member.

According to an eighth aspect, there is provided the multi-position support of any of aspects 1-7, wherein the support surface meets the intermediate surface at another interface that is at an oblique angle to sides of the primary base.

According to a ninth aspect, there is provided the multi-position support of aspect 8, wherein the oblique angles of both interfaces to the sides of the primary base are about the same.

According to a tenth aspect, a multi-position support for a multi-layer cell culture apparatus, the multi-position support comprising: a primary base that rests against a support member in an upright configuration; a support surface that is offset vertically from the primary base in the upright configuration, the support surface supporting the multi-layer cell culture apparatus with the multi-layer cell culture apparatus located thereon; and an intermediate surface that extends between the primary base and the support surface, wherein the intermediate surface meets the primary base at an interface that extends at an oblique angle to sides of the primary base; wherein the multi-position support has a tilted configuration where the multi-position support is rotated about the interface such that the cell culture apparatus is provided with a compound angle relative to horizontal.

According to an eleventh aspect, there is provided the multi-position support of aspect 10, wherein the multi-position support has the tilted configuration where the multi-position support is rotated about the interface such that the first support surface is closer to the support member than in the upright configuration with the support surface supporting the multi-layer cell culture apparatus thereon.

According to a twelfth aspect, there is provided the multi-position support of aspect 10 or 11, wherein the first support surface meets the intermediate surface at another interface that is at an oblique angle to sides of the primary base.

According to a thirteenth aspect, there is provided the multi-position support of aspect 12, wherein the oblique angles of both interfaces to the sides of the primary base are about the same.

According to a fourteenth aspect, there is provided the multi-position support of any of aspects 10-13 further comprising a secondary base that rests against the support member in the upright configuration.

According to a fifteenth aspect, there is provided the multi-position support of aspect 14, wherein the first support surface and the second support surface lie in substantially a same plane that is oblique to the primary base.

According to a sixteenth aspect, there is provided the multi-position support of aspect 15 further comprising a handle that extends outward from the secondary base and comprising a support flange that engages a fill side of the multi-layer cell culture apparatus to constrain the multi-layer cell culture apparatus on the first and second support surfaces.

According to an seventeenth aspect, there is provided the multi-position support of aspect 16 further comprising another support flange that extends outward from the first support surface that engages a rear side of the multi-layer cell culture apparatus that is opposite the fill side.

According to a eighteenth aspect, there is provided the multi-position support of any of aspects 10-17, wherein the second support surface is spaced from the support member.

According to a nineteenth aspect, a method of changing a fill angle of a cell culture apparatus comprising multiple cell culture modules each containing multiple layers of cell culture chambers, the multiple cell culture modules being fluidly connected together by a fluid manifold and an air manifold, the method comprising: connecting the cell culture apparatus to a multi-position support, the multi-position support comprising: a primary base that rests against a support member in an upright configuration; a support surface that is offset vertically from the primary base in the upright configuration, the support surface supporting the multi-layer cell culture apparatus with the multi-layer cell culture apparatus located thereon; and an intermediate surface that extends between the primary base and the support surface, wherein the intermediate surface meets the primary base at an interface that extends at an oblique angle to sides of the primary base; wherein the multi-position support has a tilted configuration where the multi-position support is rotated about the interface such that the support surface is closer to the support member than in the upright configuration with the support surface supporting the multi-layer cell culture apparatus thereon; and filling the cell culture apparatus while supported by the multi-position support with the multi-position support in either the upright configuration or the tilted configuration.

According to a twentieth aspect, there is provided the method of aspect 19, wherein the step of filling comprises filling the cell culture apparatus while supported by the multi-position support with the multi-position support in the upright configuration.

According to a twenty-first aspect, there is provided the method of aspect 20 further comprising tilting the cell culture apparatus using the multi-position support by rotating the multi-position support about the interface.

Multi-position supports described herein support cell culture apparatuses thereon and have both an upright and a tilted configuration that place the cell culture apparatuses in different angular orientations relative to horizontal. By providing the cell culture apparatuses with different angular orientations, improved fill and empty results can be achieved in a more reliable and consistent fashion. Further, in the tilt configuration, the a fill side (front) of the cell culture devices is provided with a compound angle up where an air manifold is elevated in both top-to-bottom and front-to-rear directions.

### BRIEF DESCRIPTION OF THE DRAWINGS

FIG. 1 is a perspective view of a cell culture apparatus including manifolds, according to one or more embodiments shown and described herein;

FIG. 2 is a diagrammatic of multiple stackette layers for use with the cell culture apparatus of FIG. 1, according to one or more embodiments shown and described herein;

FIG. 3 is a side view of a multi-position support that supports the cell culture apparatus of FIG. 1 in an upright configuration, according to one or more embodiments shown and described herein;

FIG. 4 is a perspective view of the multi-position support of FIG. 3, according to one or more embodiments shown and described herein;

FIG. 5 is a plan view of the multi-position support of FIG. 4, according to one or more embodiments shown and described herein;

FIG. 6 is a side view of the multi-position support of FIG. 3 in a tilted configuration, according to one or more embodiments shown and described herein;

FIG. 7 is an end view of the multi-position support of FIG. 6 in the tilted configuration, according to one or more embodiments shown and described herein; and

FIG. 8 is a side view of multiple multi-position supports that are nested, one on top of another, according to one or more embodiments shown and described herein.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”.

The present disclosure describes multi-position supports for multi-layer cell culture apparatus. The multi-position supports may be formed by bending a plate to include a primary base that rests against a support member in an upright configuration. A support surface is provided that is offset vertically from the primary base in the upright configuration and supports a multi-layer cell culture apparatus at an angle to the support surface, or at an angle to horizontal. The multi-position supports further include an intermediate surface that extends between the primary base and the support surface. The intermediate surface meets the primary base at an interface that extends at an oblique angle to sides of the primary base. The multi-position support has a tilted configuration where the multi-position support is rotated about the interface on the support surface such that the support surface is closer to the support member than in the upright configuration with the support surface supporting the multi-layer cell culture apparatus thereon.

The multi-layer cell culture apparatus include cell culture modules that include a plurality of growth or culture surfaces in cell culture chambers coupled together via manifolds to form the cell culture devices. The cell culture modules can be further coupled to additional cell culture modules via manifolds to form stacked cell culture devices. The plurality of culture surfaces may be stacked in a multi-layer configuration. The manifold includes an integral column structure that is formed as a monolithic part of the manifold. The column structure includes an inlet port and provides at least part of a fluid flow pathway from the inlet port that is in fluid communication with the individual cell culture chambers within the cell culture modules. The manifolds and associated column structures may provide a closed system where the column structures can be connected to flexible tubing to isolate the cell culture chambers from the environment during use of the cell culture apparatuses.

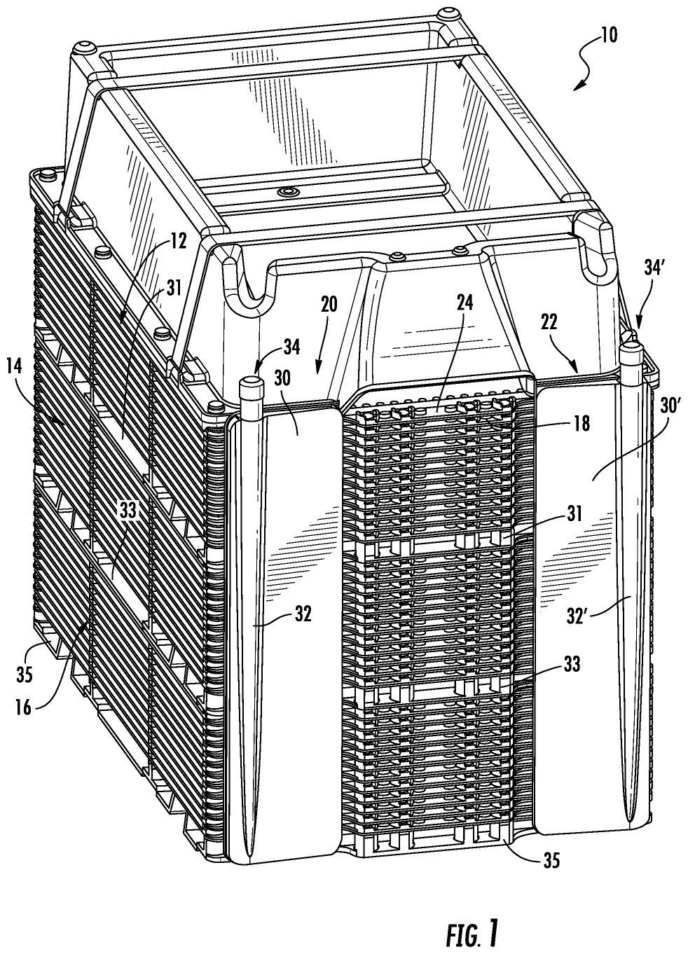

Referring to FIG. 1, a cell culture apparatus 10 includes three cell culture modules 12, 14 and 16, each containing multiple layers of cell culture chambers 18, and are stacked, one on top of the other, to form the multiple layer cell culture apparatus 10. Each cell culture module 12, 14 and 16 utilizes two manifolds 20 and 22. Liquid may enter and exit the cell culture modules 12, 14 and 16 through the first manifold 20. Thus, the first manifold 20 may be referred to as a fluid manifold. Air may enter and exit the cell culture modules 12, 14 and 16 through the second manifold 22. Thus, the second manifold 22 may be referred to as an air manifold.

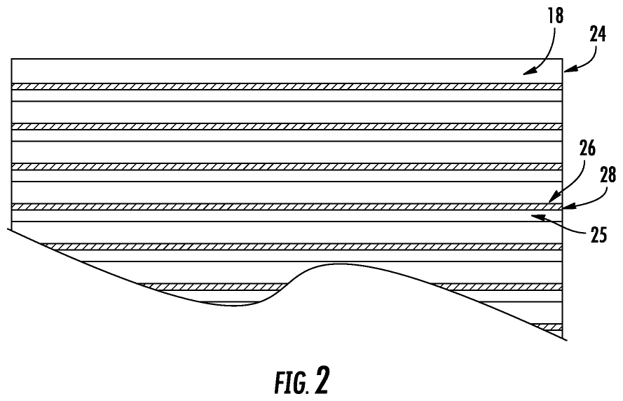

The cell culture modules 12, 14 and 16 may each include multiple stackette layers 24 that, when stacked together, form the multiple cell culture chambers 18 having tracheal spaces (air spaces) 25 there between, as shown in FIG. 2. FIG. 2 is a schematic representation of the multiple stackette layers 24 that are stacked together to form the layered cell culture chambers 18 and cell culture surfaces 26 that include a gas permeable, liquid impermeable film 28, for example, the stackette layers 24 include the tracheal spaces 25 to allow transfer of gasses between the cell culture chambers 18 and the exterior of the cell culture apparatus 10. Referring back to FIG. 1, the cell culture modules 12, 14 and 16 may be separated from one another by spacers 31, 33 and 35. The spacers 31, 33 and 35 can provide structural support for the individual cell culture modules 12, 14 and 16. In some embodiments, spacers 31 and/or 33 may be replaced by additional stackette layers 24 to provide a higher total number of cell culture chambers 18. Further, a riser volume may be provided above the cell culture module 12 to catch residual air, rather than air residing in the cell culture chambers 18.

A cell culture module, or portions thereof, as described herein may be formed from any suitable material. Preferably, materials intended to contact cells or culture media are compatible with the cells and the media. Typically, cell culture modules are formed from polymeric material. Examples of suitable polymeric materials include polystyrene, polymethylmethacrylate, polyvinyl chloride, polycarbonate, polysulfone, polystyrene copolymers, fluoropolymers, polyesters, polyamides, polystyrene butadiene copolymers, fully hydrogenated styrenic polymers, polycarbonate PDMS copolymers, and polyolefins such as polyethylene, polypropylene, polymethyl pentene, polypropylene copolymers and cyclic olefin copolymers, and the like.

In some embodiments, the culture modules contain the gas permeable, liquid impermeable film 28 to allow transfer of gasses between the cell culture chamber 18 and ultimately with the exterior of the cell culture assembly. Such culture modules can include spacers or spacer layers positioned adjacent the film, exterior to the chamber, to allow air flow between stacked units. One commercially available example of a cell culture apparatus containing such stacked gas permeable culture units is Corning's HYPERStack™ cell culture apparatus. Examples of suitable gas permeable polymeric materials useful for forming a film include polystyrene, polyethylene, polycarbonate, polyolefin, ethylene vinyl acetate, polymethylpentene, polypropylene, polytetrafluoroethylene (PTFE), or compatible fluoropolymer, a silicone rubber or copolymer, poly(styrene-butadiene-styrene) or combinations of these materials. As manufacturing and compatibility for the growth of cells permits, various polymeric materials may be utilized. Preferably the film is of a thickness that allows for efficient transfer of gas across the film. For example, a polystyrene film may be of a thickness of about 0.003 inches (about 75 micrometers), though various thicknesses are also permissive of cell growth. As such, the film may be of any thickness, preferably between about 25 and 250 micrometers, or between approximately 25 and 125 micrometers. The film allows for the free exchange of gases between the chamber of the assembly and the external environment and may take any size or shape. Preferably, the film is durable for manufacture, handling, and manipulation of the apparatus.

As mentioned above, the cell culture modules 12, 14 and 16 may be connected together using the manifolds 20 and 22. The manifold 20 includes a side wall base structure 30 and a column structure 32 that is formed as a monolithic part of the side wall base structure 30 providing a unitary manifold 20. The column structure 32 includes a barb structure 34 and provides at least part of a fluid flow pathway from the barb structure 34 that is in fluid communication with the individual cell culture chambers 18 within the cell culture modules 12, 14 and 16. The manifold 20 may be configured to allow filling and emptying of the cell culture chambers 18.

The manifold 22 also includes a side wall base structure 30′ and a column structure 32′ that is formed as a monolithic part of the side wall base structure 30′ providing a unitary manifold 22. The column structure 32′ includes a barb structure 34′ and provides at least part of a fluid flow pathway from the individual cell culture chambers 18 within the cell culture modules 12, 14 and 16 to the barb structure 34′. The manifold 22 may be configured to allow filling and emptying of the cell culture chambers 18 by allowing air to enter and exit the cell culture apparatus 10. In some embodiments, the column structure 32′ may be offset from the illustrated location in order to control media flow into the column structure 32′.

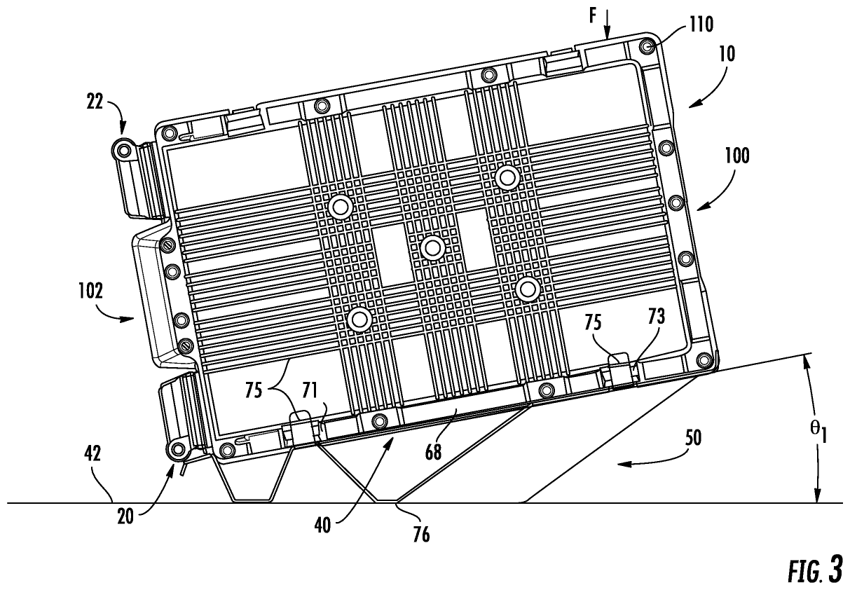

Referring to FIG. 3, the cell culture apparatus 10 may be filled and emptied with the cell culture apparatus lying on a side 40 and tilted, as illustrated by FIG. 3. The cell culture apparatus 10 may be reliably positioned on the side 40 at a predetermined tilt angle θ₁ (e.g., between about 10 degrees and about 12 degrees) relative to a support member 42 or horizontal using a multi-position support 50. The side 40 closest to the fluid manifold 20 is placed on the multi-position support 50 such that the fluid manifold 20 is lower than the air manifold 22. As will be described in greater detail below, the multi-position support 50 can be tilted between an upright configuration (as shown by FIG. 3) and a tilted configuration to position the cell culture apparatus 10 at different angles relative to horizontal.

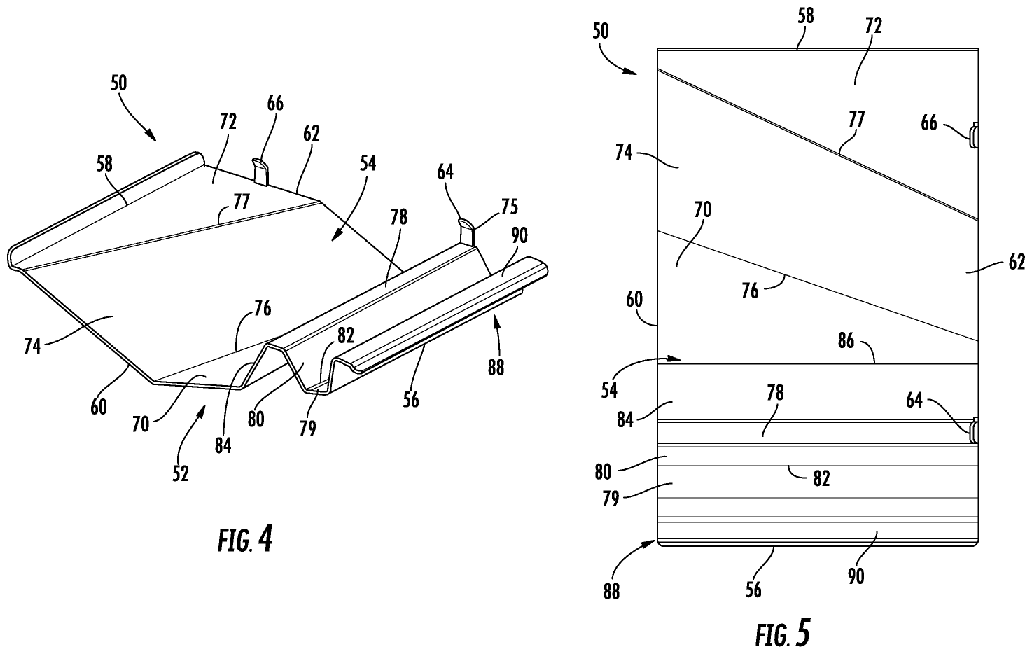

Referring to FIGS. 4 and 5, the multi-position support 50 is illustrated in isolation and is formed as a monolithic, bent plate that includes a bottom 52, a top 54, opposite ends 56 and 58 and opposite sides 60 and 62. At side 62, the multi-position support 50 includes location tabs 64 and 66 that engage a bottom edge 68 of the cell culture apparatus 10 (FIG. 3) with the multi-position support 50 in an upright, standing position and helps to hold the cell culture apparatus 10 in place on the multi-position support 50. In some embodiments, the bottom edge 68 of the cell culture apparatus 10 may be provided with recessed features 71 and 73 that are sized and located to receive the location tabs 64 and 66. The location tabs 64 and 66 may include a bend 75 that can be used to grip the bottom edge 68 and inhibit side-to-side movement of the cell culture apparatus 10 off of the multi-position support 50.

The multi-position support 50 includes a primary base 70 that rests against a support member (e.g., a table) with the multi-position support 50 in an upright configuration as shown. A primary support surface 72 is provided that is offset vertically from the primary base 70 in the upright configuration and supports the cell culture apparatus 10 thereon. The multi-position support 50 further includes an intermediate surface 74 that extends between the primary base 70 and the primary support surface 72. The intermediate surface 74 meets the primary base 70 at an interface 76 formed as a bend that extends at an oblique angle to the sides 60 and 62 of the multi-position support 50. The intermediate surface 74 also meets the primary support surface 72 at an interface 77 formed as a bend that extends at an oblique angle to the sides 60 and 62. In some embodiments, the oblique angles of the interfaces 76 and 77 are about the same (e.g., within five degrees) relative to the sides 60 and 62 or they may be different.

The multi-position support 50 further includes a secondary base 79 that rests against the support member with the multi-position support 50 in the upright configuration. A secondary support surface 78 is provided that is offset vertically from the secondary base 79 in the upright configuration and supports the cell culture apparatus 10 thereon. The secondary support surface 78 and the primary support surface 72 lie in a same plane that is at an angle to horizontal and is also oblique to the primary base 70 and the secondary base 79. The multi-position support 50 further includes another intermediate surface 80 that extends between the secondary base 79 and the secondary support surface 78. The intermediate surface 80 meets the secondary base 79 at an interface 82 formed as a bend that extends perpendicular to the sides 60 and 62 of the multi-position support 50. Another intermediate surface 84 extends between the primary base 70 and the secondary support surface 78. The intermediate surface 84 meets the primary base 70 at an interface 86 formed as a bend that also extends perpendicular to the sides 60 and 62. A handle feature 88 is provided at the end 56. The handle feature 88 can also include a support flange 90 that is offset vertically from the secondary base 79 in the upright configuration and supports the cell culture apparatus 10 thereon. The end 58 is provided with a support flange 94 that extends vertically outward from the primary support surface 72 and is used to hold the cell culture device 10 on the primary support surface 72.

FIG. 3 illustrates the multi-position support 50 with the cell culture apparatus 10 supported thereon in the upright configuration. In the upright configuration, the cell culture apparatus 10 has a rear 100 that is more elevated than a front 102 at the angle θ₁ (between 10 degrees and 12 degrees) to horizontal. However, the top to bottom angle is parallel (zero degrees) to horizontal. This upright configuration may place the cell culture apparatus 10 in an initial fill position to begin filling the cell culture apparatus 10 where the front 102 is lower than the rear 100 thereby providing a gentler fill angle, which can reduce foaming in the fluid and promote air evacuation through the air manifold and filter connected thereto.

As the cell culture apparatus 10 is being filled with the multi-position support 50 in the upright configuration, the fluid level within the cell culture apparatus 10 rises toward the air manifold 22 and toward the filter that is connected to the air manifold. Wetting of the filter can reduce airflow rate out of the cell culture apparatus 10 thereby pressurizing the interior, which can lead to an undesirable environment within the cell culture apparatus 10. To reduce a likelihood that fluid reaches the filter, the multi-position support 50 is provided with a tilted configuration where the multi-position support 50 along with the cell culture apparatus 10 is rotated without lifting either the multi-position support 50 or the cell culture apparatus 10. The multi-position support 50 along with the cell culture apparatus 10 is tilted simply by applying a force F to a rear corner 110 of the cell culture apparatus 10, which causes the multi-position support 50 and the cell culture apparatus 10 to rotate about the interface 76. Because the interface 76 extends at the oblique angle to the sides 60 and 62 of the multi-position support 50, the tilting changes both the front to rear angle and the top to bottom angle to increase the elevation of a top of the air manifold where the filter is connected.

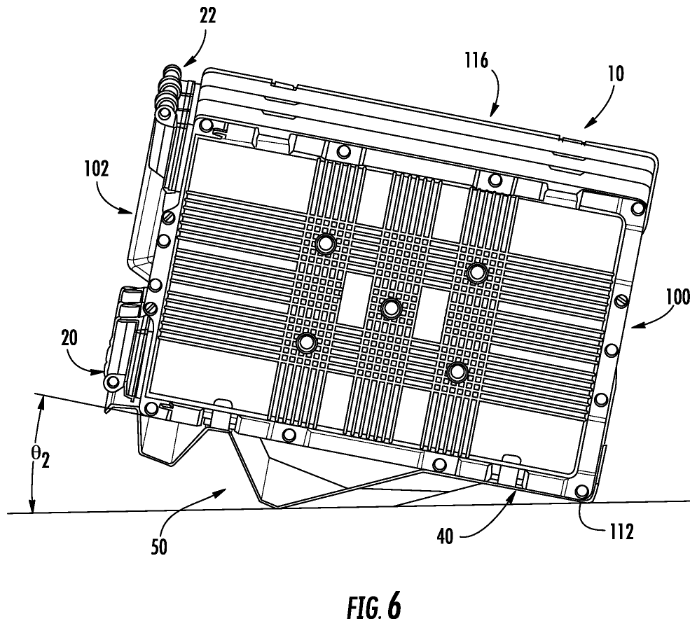

In particular, referring to FIG. 6, the multi-position support 50 and the cell culture apparatus 10 are shown in the tilted configuration where the front 102 is now more elevated than the rear 100 providing an angle θ₂ (between 11 degrees and 13 degrees) to horizontal. As can be seen, a corner 112 between the side 40 and the rear 100 of the cell culture apparatus 10 rests against the support member in the tilted configuration. Referring to FIG. 7, a top 116 is more elevated than the bottom 114 at an angle θ₃ (between seven degrees and nine degrees) to horizontal. The tilted configuration thereby provides the multi-position support 50 and the cell culture apparatus 10 with the compound angle of both θ₂ (front to rear) and θ₃ (top to bottom), which may be referred to as an end fill position. Once the cell culture apparatus 10 is filled, the side 60 of the multi-position support 50 nearest the top 116 of the cell culture apparatus 10 may be rotated upward until the cell culture apparatus 10 is in an upright, standing position. Thus, the cell culture apparatus 10 can be manipulated throughout the fill process using only the multi-position support 50 without any need for lifting the cell culture apparatus 10 from the multi-position support 50. Emptying the cell culture apparatus 10 can be performed in a reverse order.

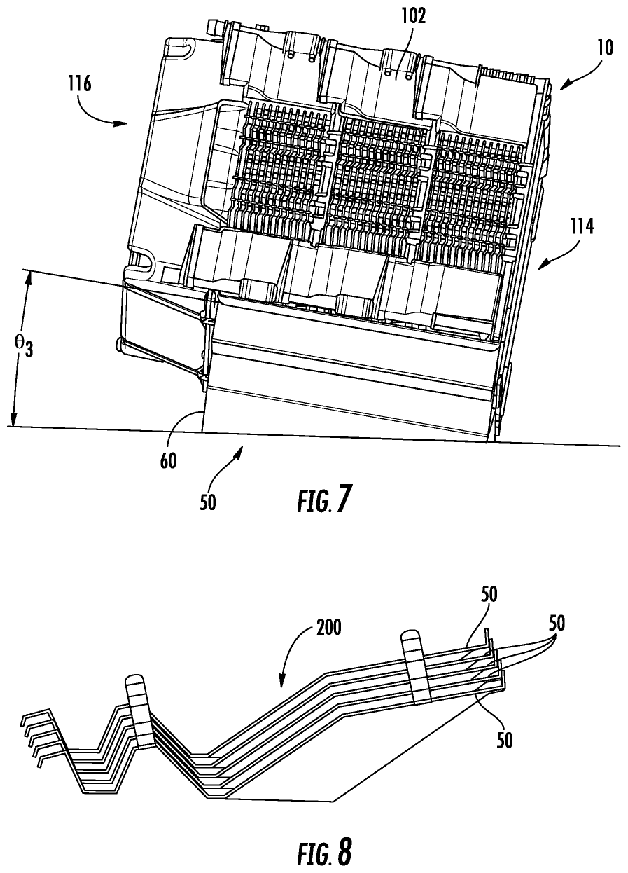

The above-described multi-position supports can be used to manipulate cell culture apparatuses without any need for handling the cell culture apparatuses separately from the multi-position supports during a fill or empty operation. The multi-position supports can thereby increase process efficiency and save users time due to higher fill and empty rates as well as from simple quick angle change procedures. The multi-position supports may further provide clear and concise control protocols which can reduce errors, reduce the possibility of product failure and/or damage, reduce angle variations due to method cradling using the multi-position supports and fixed tilt angles. Providing the multi-position supports with a compound tilt angle reduces the change of wetting out the filter attached to the air manifold. In some embodiments, the multi-position devices may be formed of stainless steel, which can provide increased durability and meet good manufacturing practices (GMP). Referring briefly to FIG. 8, the multi-position supports may be stacked into stacks 200 to minimize lab space when not in use. The multi-position apparatuses may be formed from a sheet material on a metal brake to reduce manufacturing costs. Modifications can be made without incurring significant costs for re-tooling.

Thus, embodiments of MULTI-POSITION SUPPORTS FOR CELL CULTURE APPARATUSES are disclosed. One skilled in the art will appreciate that the cell culture apparatuses and methods described herein can be practiced with embodiments other than those disclosed. The disclosed embodiments are presented for purposes of illustration and not limitation.

What is claimed is:

1. A multi-position support for a multi-layer cell culture apparatus, the multi-position support comprising:
a primary base that rests against a support member in an upright configuration;
a support surface that is offset vertically from the primary base in the upright configuration, the support surface supporting the multi-layer cell culture apparatus with the multi-layer cell culture apparatus located thereon; and
an intermediate surface that extends between the primary base and the support surface, wherein the intermediate surface meets the primary base at an interface that extends at an oblique angle to sides of the primary base;
wherein the multi-position support has a tilted configuration where the multi-position support is rotated about the interface such that the support surface is closer to the support member than in the upright configuration with the support surface supporting the multi-layer cell culture apparatus thereon.

2. The multi-position support of claim 1, further comprising a secondary base that rests against the support member in the upright configuration.

3. The multi-position support of claim 2, wherein the support surface is a first support surface, the multi-position support further comprising a second support surface located between the primary base and the secondary base, the second support surface supporting the multi-layer cell culture apparatus with the multi-layer cell culture apparatus located thereon.

4. The multi-position support of claim 3, wherein the first support surface and the second support surface lie in substantially a same plane that is oblique to the primary base.

5. The multi-position support of claim 4, further comprising a handle that extends outward from the secondary base and comprising a support flange that engages a fill side of the multi-layer cell culture apparatus to constrain the multi-layer cell culture apparatus on the first and second support surfaces.

6. The multi-position support of claim 5, further comprising another support flange that extends outward from the first support surface that engages a rear side of the multi-layer cell culture apparatus that is opposite the fill side.

7. The multi-position support of claim 4, wherein the second support surface is spaced from the support member.

8. The multi-position support of claim 1, wherein the support surface meets the intermediate surface at another interface that is at an oblique angle to sides of the primary base.

9. The multi-position support of claim 8, wherein the oblique angles of both interfaces to the sides of the primary base are about the same.

10. A multi-position support for a multi-layer cell culture apparatus, the multi-position support comprising:
a primary base that rests against a support member in an upright configuration;
a support surface that is offset vertically from the primary base in the upright configuration, the support surface supporting the multi-layer cell culture apparatus with the multi-layer cell culture apparatus located thereon; and
an intermediate surface that extends between the primary base and the support surface, wherein the intermediate surface meets the primary base at an interface that extends at an oblique angle to sides of the primary base;
wherein the multi-position support has a tilted configuration where the multi-position support is rotated about the interface such that the cell culture apparatus is provided with a compound angle relative to horizontal.

11. The multi-position support of claim 10, wherein the multi-position support has the tilted configuration where the multi-position support is rotated about the interface such that the first support surface is closer to the support member than in the upright configuration with the support surface supporting the multi-layer cell culture apparatus thereon.

12. The multi-position support of claim 10, wherein the support surface meets the intermediate surface at another interface that is at an oblique angle to sides of the primary base.

13. The multi-position support of claim 12, wherein the oblique angles of both interfaces to the sides of the primary base are about the same.

14. The multi-position support of claim 10 further comprising a secondary base that rests against the support member in the upright configuration.

15. The multi-position support of claim 14, wherein the first support surface and the second support surface lie in substantially a same plane that is oblique to the primary base.

16. The multi-position support of claim 15 further comprising a handle that extends outward from the secondary base and comprising a support flange that engages a fill side of the multi-layer cell culture apparatus to constrain the multi-layer cell culture apparatus on the first and second support surfaces.

17. The multi-position support of claim 16 further comprising another support flange that extends outward from the first support surface that engages a rear side of the multi-layer cell culture apparatus that is opposite the fill side.

18. The multi-position support of claim 10, wherein the second support surface is spaced from the support member.

19. A method of changing a fill angle of a cell culture apparatus comprising multiple cell culture modules each containing multiple layers of cell culture chambers, the multiple cell culture modules being fluidly connected together by a fluid manifold and an air manifold, the method comprising:
connecting the cell culture apparatus to a multi-position support, the multi-position support comprising:
a primary base that rests against a support member in an upright configuration;
a support surface that is offset vertically from the primary base in the upright configuration, the support surface supporting the multi-layer cell culture apparatus with the multi-layer cell culture apparatus located thereon; and
an intermediate surface that extends between the primary base and the support surface, wherein the intermediate surface meets the primary base at an interface that extends at an oblique angle to sides of the primary base;
wherein the multi-position support has a tilted configuration where the multi-position support is rotated about the interface such that the support surface is closer to the support member than in the upright configuration with the support surface supporting the multi-layer cell culture apparatus thereon; and
filling the cell culture apparatus while supported by the multi-position support with the multi-position support in either the upright configuration or the tilted configuration.

20. The method of claim 19, wherein the step of filling comprises filling the cell culture apparatus while supported by the multi-position support with the multi-position support in the upright configuration.

21. The method of claim 20, further comprising tilting the cell culture apparatus using the multi-position support by rotating the multi-position support about the interface.
